(19) United States
(12) Patent Application Publication
(10) Pub. No.: US 2025/0281706 A1
(43) Pub. Date: Sep. 11, 2025

(54) SILENCER FOR CPAP DEVICE AND CPAP DEVICE

(71) Applicant: Murata Manufacturing Co., Ltd., Nagaokakyo-shi (JP)

(72) Inventor: Hiroaki WADA, Nagaokakyo-shi (JP)

(21) Appl. No.: 19/217,116

(22) Filed: May 23, 2025

Related U.S. Application Data
(63) Continuation of application No. PCT/JP2023/037878, filed on Oct. 19, 2023.

(30) Foreign Application Priority Data
Nov. 29, 2022 (JP) 2022-190489

(51) Int. Cl. A61M 16/00

(52) U.S. Cl. CPC A61M 16/0003; A61M 16/0066

(57) ABSTRACT

A silencer for a CPAP device comprises a circulation channel through which air can be circulated and a first silencing chamber to which pressure fluctuations of the air in the circulation channel can be transmitted. The circulation channel includes a first flow channel, a second flow channel, and a third flow channel that extend along a first axis. The circulation channel further includes a first connecting flow channel that connects an end of the first flow channel on a first positive direction side and an end of the second flow channel on the first positive direction side, and a second connecting flow channel that connects an end of the second flow channel on a first negative direction side and an end of the third flow channel on the first negative direction side.

### CROSS REFERENCE TO RELATED APPLICATION

This is a continuation of International Application No. PCT/JP2023/037878 filed on Oct. 19, 2023 which claims priority from Japanese Patent Application No. 2022-190489 filed on Nov. 29, 2022. The contents of these applications are incorporated herein by reference in their entireties.

### BACKGROUND OF THE DISCLOSURE

### Field of the Disclosure

The present disclosure relates to a silencer for a continuous positive airway pressure (CPAP) device and a CPAP device.

### Description of the Related Art

International Publication No. WO2019/189126A1 describes a CPAP device that includes a main unit and a sub-unit. The main unit incorporates a blower. The sub-unit incorporates a silencer. The silencer includes a flow passage of air for the blower, and a resonance tube branched from the flow passage. The resonance tube is a side branch type resonator.

### BRIEF SUMMARY OF THE DISCLOSURE

In a CPAP device, such as that of International Publication No. WO2019/189126A1, the amount of air sent from the blower needs to be changed in accordance with breathing of a user. Therefore, the CPAP device changes a rotational speed or the like of a fan of the blower in accordance with the breathing of the user. Accordingly, in the CPAP device, noise may occur in various frequency bands. The CPAP device of International Publication No. WO2019/189126A1 has a noise reduction effect on a specific frequency that corresponds to the shape of the resonance tube. However, the noise reduction effect of the CPAP device of International Publication No. WO2019/189126A1 may be limited in a frequency band outside the specific frequency.

A silencer for a CPAP device is provided to solve the above problem. In one general aspect, the silencer includes a flow passage through which air flows in accordance with operation of a blower, and a noise cancellation chamber that allows for transmission of pressure fluctuations of the air in the flow passage. The flow passage includes a first flow passage, a second flow passage, a third flow passage, a first connection passage, and a second connection passage. The first flow passage extends along a first axis. The second flow passage extends along the first axis and is arranged next to the first flow passage in a direction orthogonal to the first axis. The third flow passage extends along the first axis and is arranged next to the second flow passage in the direction orthogonal to the first axis. The first connection passage connects an end of the first flow passage in a positive direction and an end of the second flow passage in the positive direction. The positive direction is one of two directions parallel to the first axis. The second connection passage connects an end of the second flow passage in a negative direction and an end of the third flow passage in the negative direction. The negative direction is another one of the two directions parallel to the first axis and is opposite to the positive direction. A maximum dimension of the first connection passage in a direction in which the second flow passage is arranged next to the third flow passage differs from a maximum dimension of the second connection passage in the direction in which the second flow passage is arranged next to the third flow passage. The noise cancellation chamber is located between the second flow passage and the third flow passage in the direction in which the second flow passage is arranged next to the third flow passage. The noise cancellation chamber is connected to the second connection passage through an opening included in a wall that separates the noise cancellation chamber and the second connection passage.

In another general aspect, a CPAP device includes a blower that blows air, and a silencer. The silencer includes a flow passage through which air flows in accordance with operation of the blower, and a noise cancellation chamber that allows for transmission of pressure fluctuations of the air in the flow passage. The flow passage includes a first flow passage, a second flow passage, a third flow passage, a first connection passage, and a second connection passage. The first flow passage extends along a first axis. The second flow passage extends along the first axis and is arranged next to the first flow passage in a direction orthogonal to the first axis. The third flow passage extends along the first axis and is arranged next to the second flow passage in the direction orthogonal to the first axis. The first connection passage connects an end of the first flow passage in a positive direction and an end of the second flow passage in the positive direction. The positive direction is one of two directions parallel to the first axis. The second connection passage connects an end of the second flow passage in a negative direction and an end of the third flow passage in the negative direction. The negative direction is another one of the two directions parallel to the first axis and is opposite to the positive direction. A maximum dimension of the first connection passage in a direction in which the second flow passage is arranged next to the third flow passage differs from a maximum dimension of the second connection passage in the direction in which the second flow passage is arranged next to the third flow passage. The noise cancellation chamber is located between the second flow passage and the third flow passage in the direction in which the second flow passage is arranged next to the third flow passage. The noise cancellation chamber is connected to the second connection passage through an opening included in a wall that separates the noise cancellation chamber and the second connection passage.

With the above structure, the maximum dimension of the first connection passage in the direction in which the second flow passage is arranged next to the third flow passage differs from the maximum dimension of the second connection passage in the direction in which the second flow passage is arranged next to the third flow passage. Such a difference in the dimension of the connection passages allows for cancellation of noise in different frequency bands in the connection passages. Consequently, noise in various frequency bands is reduced. Furthermore, the silencer of the CPAP device includes the noise cancellation chamber, and the opening continuous with the noise cancellation chamber is arranged at a position where the flow passage is bent. This facilitates propagation of pressure fluctuations of air, or noise, into the noise cancellation chamber. Consequently, noise is effectively cancelled in the noise cancellation chamber.

Noise in various frequency bands may be reduced or cancelled.

### BRIEF DESCRIPTION OF THE SEVERAL VIEWS OF THE DRAWINGS

FIG. 1 is a schematic diagram showing a CPAP device in use.

FIG. 2 is an exploded perspective view of the CPAP device.

FIG. 3 is a schematic diagram showing the configuration of the CPAP device.

FIG. 4 is a diagram of the CPAP device shown in FIG. 2, as seen through a bottom surface.

### DETAILED DESCRIPTION OF THE DISCLOSURE

### <Embodiment of Silencer for CPAP Device>

An embodiment of a silencer for a continuous positive airway pressure (CPAP) device will now be described with reference to the drawings. Depiction of elements in the drawings may be exaggerated for clarity, illustration, or convenience. The elements may not be drawn to scale, and the relative size and proportions of the elements may differ between the drawings.

### (Structure of CPAP Device)

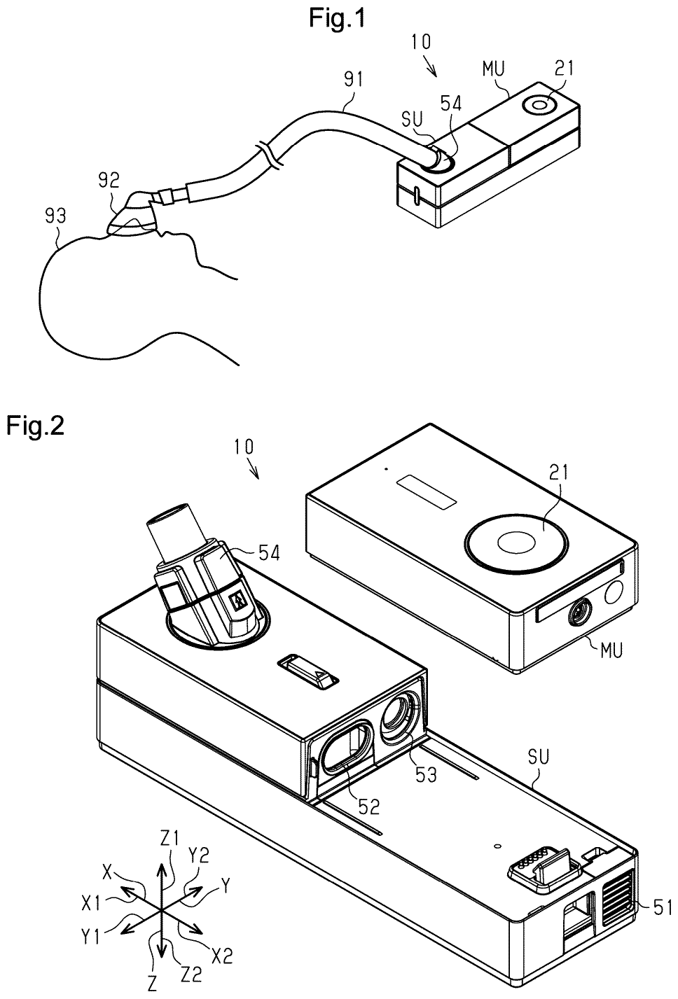

The overall configuration of the CPAP device 10 will be described. As shown in FIGS. 1 and 2, a CPAP device 10 includes a main unit MU and a sub-unit SU. In a used state in which the main unit MU is attached to the sub-unit SU, that is, in a state in which the CPAP device 10 is in use, the CPAP device 10 has the form of a box as a whole.

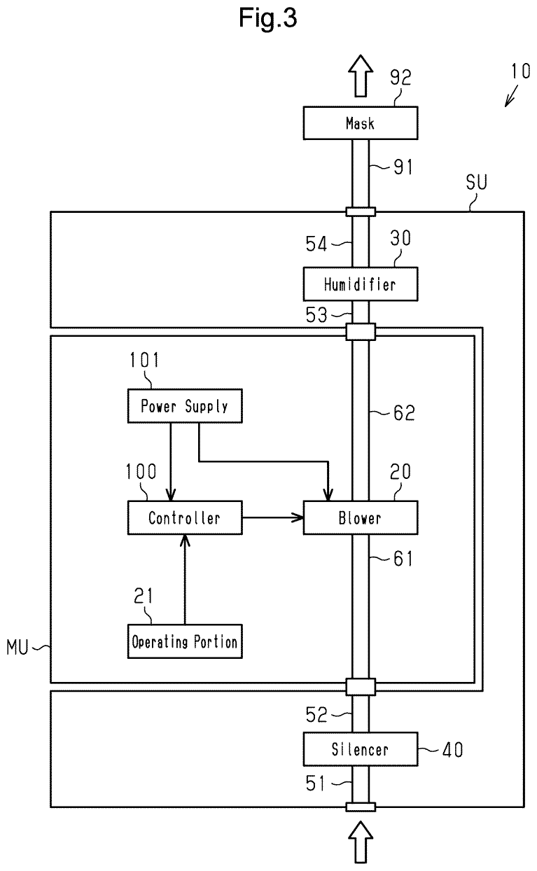

As shown in FIG. 2, the main unit MU of the CPAP device 10 is substantially box-shaped. As shown in FIG. 3, the main unit MU includes a blower 20, an operating portion 21, a controller 100, and a power supply 101.

The blower 20 accommodates a fan (not shown) that blows air. The operating portion 21 is disposed on an upper surface of the main unit MU. The operating portion 21 is used to control ON/OFF states or the like of the blower 20 via the controller 100.

The controller 100 receives a signal corresponding to an operation performed on the operating portion 21. Based on the signal received from the operating portion 21, the controller 100 outputs a control signal to the blower 20. The controller 100 controls the ON/OFF states, rotational speed, or the like of the blower 20 using the control signal.

The controller 100 may be circuitry including one or more processors that run computer programs (software) to execute various processes, one or more dedicated hardware circuits such as application-specific integrated circuits (ASICs) that execute at least some of the various processes, or a combination of the above.

The processor includes a central processing unit (CPU) and memory, such as random-access memory (RAM), read-only memory (ROM), or the like. The memory stores program codes or instructions configured to cause the CPU to execute processes. The memory, which is a computer readable medium, may be any available medium accessible by a versatile or dedicated computer.

The power supply 101 supplies power to, for example, the controller 100 and the blower 20. Specifically, the power supply 101 includes a battery. Specifically, the power supply 101 includes a rechargeable battery that can be charged repeatedly.

The main unit MU includes an upstream passage 61 and a downstream passage 62. The upstream passage 61 and the downstream passage 62 are arranged inside the main unit MU. The upstream passage 61 and the downstream passage 62 are connected to the blower 20. The blower 20 draws air from the upstream passage 61 and blows the air to the downstream passage 62.

As shown in FIG. 2, the sub-unit SU of the CPAP device 10 is L-shaped in side view taken in a direction orthogonal to a longitudinal direction. When the main unit MU is attached to the sub-unit SU, the CPAP device 10 becomes substantially box-shaped, as described above. The sub-unit SU is attachable to and detachable from the main unit MU.

As shown in FIG. 3, the sub-unit SU includes a humidifier 30 and a silencer 40. That is, the sub-unit SU corresponds to the silencer for a CPAP device. The humidifier 30 is configured to store water. The air blown by the fan of the blower 20 may flow into the humidifier 30. The silencer 40 is configured to reduce noise generated by operation of the blower 20. The shape or the like of the silencer 40 will be described in detail later.

As shown in FIG. 2, an axis extending in the longitudinal direction of the sub-unit SU is referred to as a first axis X. An axis orthogonal to the first axis X is referred to as a second axis Y. In the present embodiment, the second axis Y extends in a transverse direction of the sub-unit SU. An axis orthogonal to the first axis X and the second axis Y is referred to as a third axis Z. One of two directions parallel to the first axis X is referred to as a first positive direction X1, and the other one of the two directions parallel to the first axis X is referred to as a first negative direction X2. The first negative direction X2 is opposite to the first positive direction X1. One of two directions parallel to the second axis Y is referred to as a second positive direction Y1, and the other one of the two directions parallel to the second axis Y is referred to as a second negative direction Y2. The second negative direction Y2 is opposite to the second positive direction Y1. One of two directions parallel to the third axis Z is referred to as a third positive direction Z1, and the other one of the two directions parallel to the third axis Z is referred to as a third negative direction Z2. The third negative direction Z2 is opposite to the third positive direction Z1.

As shown in FIG. 3, the sub-unit SU includes a first inflow passage 51, a first outflow passage 52, a second inflow passage 53, and a second outflow passage 54.

The first inflow passage 51 is arranged inside the sub-unit SU. The first inflow passage 51 has a first end that is open to the outside of the sub-unit SU. The first inflow passage 51 has a second end that is connected to the silencer 40. The first outflow passage 52 is arranged inside the sub-unit SU. The first outflow passage 52 has a first end that is connected to the silencer 40. The first outflow passage 52 has a second end that is open to the outside of the sub-unit SU.

The second inflow passage 53 is arranged inside the sub-unit SU. The second inflow passage 53 has a first end that is open to the outside of the sub-unit SU. The second inflow passage 53 has a second end that is connected to the humidifier 30. The second outflow passage 54 is arranged inside the sub-unit SU. The second outflow passage 54 has a first end that is connected to the humidifier 30. The second outflow passage 54 has a second end that is open to the outside of the sub-unit SU.

As shown in FIG. 1, the CPAP device 10 includes a hose 91 and a mask 92. The hose 91 has a first end connected to the second end of the second outflow passage 54. The hose 91 has a second end connected to the mask 92. The mask 92 is attached to a user 93 so as to cover the nose or the mouth of the user 93. Although not illustrated, the first end of the hose 91 may be connected to the downstream passage 62 of the main unit MU.

### (Flow Passage of Air in Used State)

As described above, the sub-unit SU is attachable to the main unit MU. In a state in which the sub-unit SU is attached to the main unit MU, the passages of the main unit MU and the passages of the sub-unit SU are all connected to form a single flow passage. Specifically, as shown in FIG. 3, the upstream passage 61 of the main unit MU is connected to the second end of the first outflow passage 52 of the sub-unit SU. Further, the downstream passage 62 of the main unit MU is connected to the first end of the second inflow passage 53 of the sub-unit SU. Thus, when the blower 20 is operated, air is drawn from the first inflow passage 51 and flows through the silencer 40, the first outflow passage 52, and the upstream passage 61 to the blower 20. Then, the air is blown by the blower 20 and flows through the downstream passage 62, the second inflow passage 53, the second outflow passage 54, and the hose 91 to the mask 92.

Although not illustrated, the CPAP device 10 may also be used in a state in which the sub-unit SU is detached from the main unit MU. In this case, air flows through the upstream passage 61, the blower 20, the downstream passage 62, and the hose 91 to the mask 92.

### (Silencer of CPAP Device)

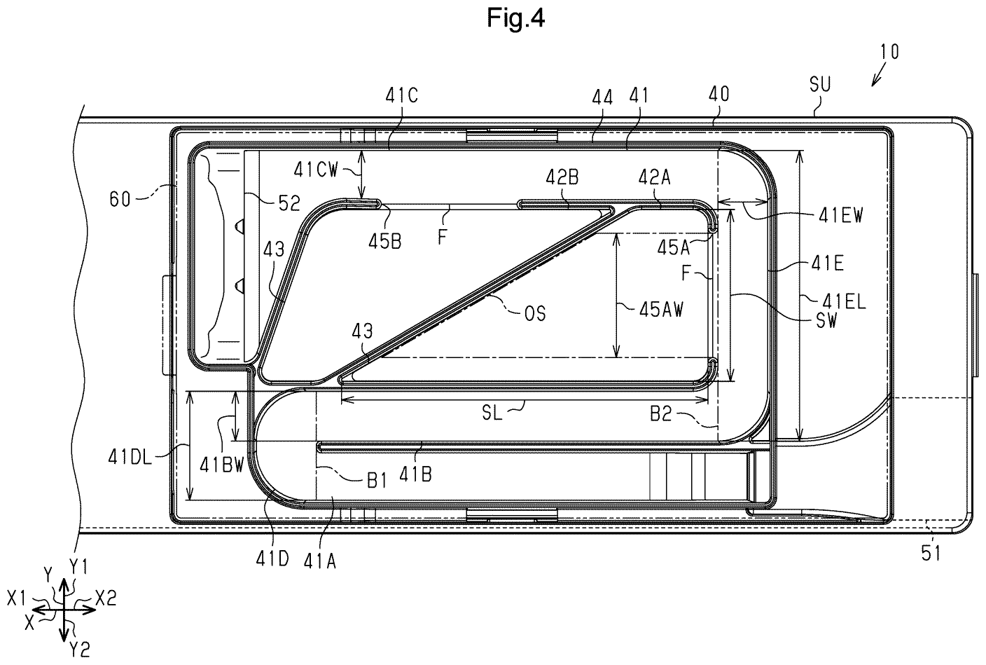

The structure of the silencer 40 and its periphery will now be described. As shown in FIG. 4, the silencer 40 includes a flow passage of air arranged inside the sub-unit SU. In FIG. 4, the sub-unit SU is seen through a bottom lid 60 that seals a side of the sub-unit SU oriented in the third negative direction Z2. The bottom lid 60 is indicated by the double-dashed lines.

The above-described first inflow passage 51 extends along the first axis X inside the sub-unit SU. The first end of the first inflow passage 51 is open in a surface of the sub-unit SU that is oriented in the first negative direction X2. The silencer 40 of the CPAP device 10 includes a flow passage 41. The flow passage 41 is a passage through which air flows in accordance with operation of the blower 20. The flow passage 41 is defined by walls 44. An upstream end of the flow passage 41 is connected to the second end of the first inflow passage 51.

The flow passage 41 includes a first flow passage 41A, a second flow passage 41B, a third flow passage 41C, a first connection passage 41D, and a second connection passage 41E. The first flow passage 41A extends along the first axis X. An end of the first flow passage 41A in the first negative direction X2 is connected to the second end of the first inflow passage 51. The second flow passage 41B extends along the first axis X. The second flow passage 41B is arranged next to the first flow passages 41A in the second positive direction Y1. The second flow passage 41B and the first flow passage 41A are arranged side by side with a single wall 44 in between.

The third flow passage 41C extends along the first axis X. The third flow passage 41C is arranged next to the second flow passage 41B in the second positive direction Y1. The third flow passage 41C and the second flow passage 41B are spaced apart from each other in a direction parallel to the second axis Y.

The first connection passage 41D extends along the second axis Y. An end of the first connection passage 41D in the second negative direction Y2 is connected to an end of the first flow passage 41A in the first positive direction X1. An end of the first connection passage 41D in the second positive direction Y1 is connected to an end of the second flow passage 41B in the first positive direction X1. Thus, the first flow passage 41A, the first connection passage 41D, and the second flow passage 41B form a U-shaped passage as a whole.

The second connection passage 41E extends along the second axis Y. An end of the second connection passage 41E in the second negative direction Y2 is connected to an end of the second flow passage 41B in the first negative direction X2. An end of the second connection passage 41E in the second positive direction Y1 is connected to an end of the third flow passage 41C in the first negative direction X2. Thus, the second flow passage 41B, the second connection passage 41E, and the third flow passage 41C form a U-shaped passage as a whole.

A maximum dimension of the first connection passage 41D in a direction parallel to the second axis Y is referred to as a first connection passage length 41DL. A maximum dimension of the second connection passage 41E in a direction parallel to the second axis Y is referred to as a second connection passage length 41EL. The first connection passage length 41DL differs from the second connection passage length 41EL. Specifically, the second connection passage length 41EL is greater than the first connection passage length 41DL due to the space between the second flow passage 41B and the third flow passage 41C.

The above-described first outflow passage 52 extends along the third axis Z inside the sub-unit SU. The first end of the first outflow passage 52 is connected to an end of the third flow passage 41C in the first positive direction X1. The second end of the first outflow passage 52 is open in a surface of the sub-unit SU that is oriented in the first negative direction X2. More specifically, the second end of the first outflow passage 52 is open in an inner surface of the L-shape of the substantially L-shaped sub-unit SU.

When the blower 20 of the CPAP device 10 is operated, air flows through the silencer 40. Specifically, air flows through the first flow passage 41A, the first connection passage 41D, the second flow passage 41B, the second connection passage 41E, and the third flow passage 41C in this order. Therefore, the second connection passage 41E is located closer to the blower 20 than the first connection passage 41D is with respect to an airflow direction.

An imaginary line extending along the center of a passage from one end to the other end serves as a center axis of the passage. If the center axis of the passage and the first axis X form an acute angle that is 0 degrees or greater and less than 45 degrees, it is considered that the passage extends along the first axis X. In the present embodiment, each of the first to third flow passages 41A to 41C forms substantially 0 degrees with the first axis X. The same also applies to a relationship between the second axis Y and a flow passage and a relationship between the third axis Z and a flow passage.

The boundary between the first flow passage 41A and the first connection passage 41D and the boundary between the second flow passage 41B and the first connection passage 41D are defined as described below. An imaginary plane orthogonal to the first axis X, which is parallel to the center axis of the first flow passage 41A, is referred to as a first imaginary plane B1. The first imaginary plane B1 is in contact with the wall 44 that separates the first flow passage 41A and the second flow passage 41B. Further, the first imaginary plane B1 is located at an end of the wall 44 toward the first connection passage 41D. The portion extending from the first imaginary plane B1 in the first positive direction X1 corresponds to the first connection passage 41D. In the same manner, another imaginary plane orthogonal to the first axis X is referred to as a second imaginary plane B2. The second imaginary plane B2 is in contact with the wall 44 that separates the second flow passage 41B and the third flow passage 41C. Further, the second imaginary plane B2 is located at an end of the wall 44 toward the second connection passage 41E. The portion extending from the second imaginary plane B2 in the first negative direction X2 corresponds to the second connection passage 41E.

### (Noise Cancellation Chamber of Silencer)

As shown in FIG. 4, the silencer 40 of the CPAP device 10 includes a first noise cancellation chamber 42A and a second noise cancellation chamber 42B. The first noise cancellation chamber 42A and the second noise cancellation chamber 42B allow for transmission of pressure fluctuations of the air in the flow passage 41. The first noise cancellation chamber 42A and the second noise cancellation chamber 42B are defined by the walls 44.

The first noise cancellation chamber 42A is located between the second flow passage 41B and the third flow passage 41C in a direction parallel to the second axis Y. The first noise cancellation chamber 42A is connected to the second connection passage 41E through a first opening 45A included in the wall 44 that separates the first noise cancellation chamber 42A and the second connection passage 41E. Further, an open area of the first opening 45A is greater than a cross-sectional flow area of the second connection passage 41E at a portion connected to the first opening 45A. The cross-sectional flow area of the second connection passage 41E refers to a cross-sectional area of the second connection passage 41E in a direction orthogonal to the center axis of the second connection passage 41E.

The first noise cancellation chamber 42A includes an inner surface on which an opposing surface OS is defined. The opposing surface OS opposes the first opening 45A and is non-perpendicular with respect to a direction in which the first opening 45A is oriented. Specifically, the opposing surface OS is a flat surface inclined with respect to the direction in which the first opening 45A is oriented. The direction in which the first opening 45A is oriented coincides with a direction in which the inner surface of the first noise cancellation chamber 42A is viewed such that the apparent area of an opening surface of the first opening 45A is maximal. In the present embodiment, the direction in which the first opening 45A is oriented coincides with the first positive direction X1. In other words, the direction in which the first opening 45A is oriented coincides with a direction orthogonal to the opening surface of the first opening 45A. When the wall 44 that separates the first noise cancellation chamber 42A and the second connection passage 41E is orthogonally projected by a light emitted in the direction in which the first opening 45A is oriented, the portion of the inner surface of the first noise cancellation chamber 42A irradiated with the light corresponds to the opposing surface OS opposing the first opening 45A.

A maximum dimension of the first noise cancellation chamber 42A in a direction parallel to the first axis X is referred to as a first noise cancellation chamber length SL, and a maximum dimension of the first noise cancellation chamber 42A in a direction parallel to the second axis Y is referred to as a first noise cancellation chamber width SW. A maximum dimension of the second connection passage 41E in a direction parallel to the first axis X is referred to as a second connection passage width 41EW. A maximum dimension of the second flow passage 41B in a direction parallel to the second axis Y is referred to as a second flow passage width 41BW. A maximum dimension of the third flow passage 41C in a direction parallel to the second axis Y is referred to as a third flow passage width 41CW. In this case, the first noise cancellation chamber length SL is greater than the second connection passage width 41EW. The first noise cancellation chamber width SW is greater than each of the second flow passage width 41BW and the third flow passage width 41CW. A maximum dimension of the first opening 45A in a direction parallel to the second axis Y is referred to as a first opening width 45AW. The first opening width 45AW is less than the first noise cancellation chamber width SW.

The second noise cancellation chamber 42B is located between the second flow passage 41B and the third flow passage 41C in a direction parallel to the second axis Y. The second noise cancellation chamber 42B is connected to the third flow passage 41C through a second opening 45B included in the wall 44 that separates the second noise cancellation chamber 42B and the third flow passage 41C. A maximum dimension of the second opening 45B in a direction parallel to the first axis X is less than a maximum dimension of the second noise cancellation chamber 42B in a direction parallel to the first axis X. Further, an open area of the second opening 45B is greater than a cross-sectional flow area of the third flow passage 41C. The cross-sectional flow area of the third flow passage 41C is defined in the same manner as that of the second connection passage 41E.

The second noise cancellation chamber 42B includes an inner surface on which an opposing surface is defined. The opposing surface opposes the second opening 45B and is non-perpendicular with respect to a direction in which the second opening 45B is oriented. Specifically, the opposing surface on the inner surface of the second noise cancellation chamber 42B is a flat surface inclined with respect to the direction in which the second opening 45B is directed.

The maximum dimension of the second noise cancellation chamber 42B in a direction parallel to the first axis X is greater than the second connection passage width 41EW. Further, a maximum dimension of the second noise cancellation chamber 42B in a direction parallel to the second axis Y is greater than each of the second flow passage width 41BW and the third flow passage width 41CW. In the present embodiment, the maximum dimension of the second noise cancellation chamber 42B in a direction parallel to the second axis Y is equal to the first noise cancellation chamber width SW.

The silencer 40 of the CPAP device 10 includes a sound absorbing material 43 arranged in the first noise cancellation chamber 42A and the second noise cancellation chamber 42B. The sound absorbing material 43 is shaped in correspondence with the noise cancellation chambers. That is, each noise cancellation chamber is substantially filled with the sound absorbing material 43. The sound absorbing material 43 is a porous material. Specifically, the sound absorbing material 43 includes, for example, urethane foam.

The silencer 40 of the CPAP device 10 includes a film F. The film F closes the first opening 45A. The film F is thinner than the wall 44 that separates the first noise cancellation chamber 42A and the second connection passage 41E. The material of the film F is soft and has a lower elastic coefficient than the wall 44. The material of the film F includes a synthetic resin, such as polyethylene, polyethylene terephthalate, or the like. Further, another film that is not the film F, which closes the first opening 45A, closes the second opening 45B. The film is thinner than the wall 44 that separates the second noise cancellation chamber 42B and the third flow passage 41C. The material of the film is the same as that of the film F, which closes the first opening 45A.

### Advantages of the Present Embodiment

(1) In the above embodiment, the first connection passage length 41DL, which is the maximum dimension of the first connection passage 41D in a direction parallel to the second axis Y, differs from the second connection passage length 41EL, which is the maximum dimension of the second connection passage 41E in a direction parallel to the second axis Y. Such a difference in the dimension of the connection passages allows for cancellation of noise in different frequency bands in the connection passages. Consequently, noise in various frequency bands is reduced.

Further, the silencer 40 of the CPAP device 10 includes the first noise cancellation chamber 42A. The first noise cancellation chamber 42A is connected to the second connection passage 41E through the first opening 45A included in the wall 44 that separates the first noise cancellation chamber 42A and the second connection passage 41E. The opening continuous with the first noise cancellation chamber 42A is arranged at a portion where the flow passage 41 is bent. This facilitates propagation of pressure fluctuations of air, or noise, into the first noise cancellation chamber 42A. Consequently, noise is effectively cancelled in the first noise cancellation chamber 42A.

(2) In the above embodiment, the silencer 40 of the CPAP device 10 further includes the sound absorbing material 43 arranged in the first noise cancellation chamber 42A. This readily reduces noise propagated into the first noise cancellation chamber 42A. That is, noise is cancelled more effectively. Further, the sound absorbing material 43 hinders air from flowing into the first noise cancellation chamber 42A, thereby ensuring the airflow through the flow passage 41 is unobstructed. This limits the pressure loss of the air flowing through the flow passage 41. The same applies to the second noise cancellation chamber 42B.

(3) In the above embodiment, the opposing surface OS is non-perpendicular with respect to the direction in which the first opening 45A is oriented. Therefore, the opposing surface OS reflects pressure fluctuations of the air transmitted through the first opening 45A toward a location other than the first opening 45A. In other words, the reflected noise will not leak out of the first noise cancellation chamber 42A through the first opening 45A. The same applies to the second noise cancellation chamber 42B and the second opening 45B.

(4) In the above embodiment, the first noise cancellation chamber width SW and the first noise cancellation chamber length SL are both greater than each of the second flow passage width 41BW, the third flow passage width 41CW, and the second connection passage width 41EW. That is, the first noise cancellation chamber 42A has a sufficient volume. Accordingly, noise propagated into the first noise cancellation chamber 42A will remain in the first noise cancellation chamber 42A for a relatively long period. This readily reduces the noise, thereby improving the noise cancellation effect. The same applies to the second noise cancellation chamber 42B.

(5) In the above embodiment, the second connection passage 41E is located closer to the blower 20 than the first connection passage 41D is. In other words, the first noise cancellation chamber 42A is located near the blower 20, which is the source of noise. This facilitates propagation of noise into the first noise cancellation chamber 42A, thereby improving the noise cancellation effect.

(6) In the above embodiment, the open area of the first opening 45A is greater than the cross-sectional flow area of the second connection passage 41E. This increases a ratio of noise propagated into the first noise cancellation chamber 42A to noise moving straight-ahead in the flow passage 41, thereby improving the noise cancellation effect. The same applies to the second opening 45B of the second noise cancellation chamber 42B.

(7) In the above embodiment, the first opening width 45AW is less than the first noise cancellation chamber width SW. Accordingly, even when noise propagated into the first noise cancellation chamber 42A is reflected by the inner surface of the wall 44 of the first noise cancellation chamber 42A, the noise will not leak out of the first opening 45A. This improves the noise cancellation effect. The same applies to the maximum dimension of the second opening 45B in a direction parallel to the first axis X and the maximum dimension of the second noise cancellation chamber 42B in a direction parallel to the first axis X.

(8) In the above embodiment, the first noise cancellation chamber 42A includes the film F in the first opening 45A. The film F is thinner than the wall 44 that separates the first noise cancellation chamber 42A and the second connection passage 41E. Since the film F is thinner than the wall 44, noise is propagated into the first noise cancellation chamber 42A. On the other hand, air flowing through the flow passage 41 will not enter the first noise cancellation chamber 42A. This limits the pressure loss of the air flowing through the flow passage 41 without adversely affecting the noise cancellation effect. Furthermore, the sound absorbing material 43 arranged in the first noise cancellation chamber 42A becomes less likely to contact air, so that collection of odor on the sound absorbing material 43 is limited. The same applies to the second noise cancellation chamber 42B.

(9) In the above embodiment, the silencer 40 of the CPAP device 10 includes two noise cancellation chambers, namely, the first noise cancellation chamber 42A and the second noise cancellation chamber 42B. These two noise cancellation chambers reduce noise in a relatively wide frequency band.

(10) In the above embodiment, pressure fluctuations of the air in the second connection passage 41E are readily transmitted into the first noise cancellation chamber 42A, and pressure fluctuations of the air in the third flow passage 41C are readily transmitted to the second noise cancellation chamber 42B. In this manner, noise is reduced at different locations, thereby improving the noise cancellation effect.

(11) In the above embodiment, the second noise cancellation chamber 42B is located closer to the blower 20, or the source of noise, than the first noise cancellation chamber 42A is. Therefore, noise is reduced efficiently.

### Modified Examples

The above embodiment and the following modifications may be combined as long as the combined modifications remain technically consistent with each other.

The main unit MU and the sub-unit SU may have any shape. The shapes of the units may be freely designed as long as the functionality of the CPAP device 10 is implemented.

The sub-unit SU does not have to include the humidifier 30.

The CPAP device 10 may include another unit in addition to the main unit MU and the sub-unit SU. Alternatively, the CPAP device 10 may be formed as a single unit that incorporates the main unit MU and the sub-unit SU.

The silencer 40 of the CPAP device 10 may be located at a downstream side of the blower 20. For example, the downstream passage 62 of the main unit MU may be connected to the first inflow passage 51 of the sub-unit SU.

The power supply 101 is not limited to the example described in the above embodiment, as long as the power supply 101 is configured to supply power to the controller 100 and the blower 20. For example, the main unit MU may include a terminal connected to an AC adapter, and the controller 100 and the blower 20 may be supplied with power from a commercial power supply via the AC adapter.

The direction in which the first flow passage 41A is arranged next to the second flow passage 41B does not have to be the same as the direction in which the second flow passage 41B is arranged next to the third flow passage 41C. For example, the first flow passage 41A may be arranged next to the second flow passage 41B in a direction parallel to the second axis Y, and the second flow passage 41B may be arranged next to the third flow passage 41C in a direction parallel to the third axis Z.

There may be four or more flow passages that extend along the first axis X. In this case, for example, a third connection passage connects the third flow passage 41C and a fourth flow passage.

In the above embodiment, the first flow passage 41A, the second flow passage 41B, and the third flow passage 41C are specified in this order from the upstream side for illustrative purposes. However, any passage may be specified as “second flow passage” or “third flow passage”. In other words, as along as the first noise cancellation chamber 42A is formed between any two of the three passages in the above embodiment, it can be considered that the first noise cancellation chamber 42A is defined between “second flow passage” and “third flow passage”.

The shape of the flow passage 41 is not limited to the example described in the above embodiment. For example, the walls 44 defining the first flow passage 41A, the second flow passage 41B, and the third flow passage 41C do not have to be straight. For example, the walls 44 may be undulated or have a plurality of protrusions as in a suppressor. Furthermore, inner surfaces of the walls 44 of the flow passage 41 may be covered with the sound absorbing material 43.

The first opening width 45AW may be equal to the first noise cancellation chamber width SW. Even in this case, advantage (1) is obtained. Furthermore, the maximum dimension of the second opening 45B in a direction parallel to the first axis X may be equal to the maximum dimension of the second noise cancellation chamber 42B in a direction parallel to the first axis X. Even in this case, advantages (1) and (9) are obtained.

The open area of the first opening 45A may be less than or equal to the cross-sectional flow area of the second connection passage 41E. Even if the open area of the first opening 45A is relatively small, advantage (1) will be obtained as long as pressure fluctuations of the air in the flow passage 41 can be transmitted through the first opening 45A. Furthermore, the open area of the second opening 45B may be less than or equal to the cross-sectional flow area of the third flow passage 41C. For the same reason, advantages (1) and (9) are obtained.

The opposing surface OS on the inner surface of the first noise cancellation chamber 42A, which opposes the first opening 45A, may be perpendicular with respect to the direction in which the first opening 45A is oriented. Even in this case, advantage (1) is obtained. Furthermore, the opposing surface on the inner surface of the second noise cancellation chamber 42B, which opposes the second opening 45B, may be perpendicular with respect to the direction in which the second opening 45B is oriented. Even in this case, advantages (1) and (9) are obtained.

The first noise cancellation chamber length SL may be less than or equal to the second connection passage width 41EW. Furthermore, the first noise cancellation chamber width SW may be less than or equal to the second flow passage width 41BW, and the first noise cancellation chamber width SW may be less than or equal to the third flow passage width 41CW. Even if the first noise cancellation chamber 42A is relatively small, advantage (1) is obtained.

The maximum dimension of the second noise cancellation chamber 42B in a direction parallel to the first axis X may be less than or equal to the second connection passage width 41EW. Furthermore, the maximum dimension of the second noise cancellation chamber 42B in a direction parallel to the second axis Y may be less than or equal to the second flow passage width 41BW, and the maximum dimension of the second noise cancellation chamber 42B in a direction parallel to the second axis Y may be less than or equal to the third flow passage width 41CW. Even if the second noise cancellation chamber 42B is relatively small, advantages (1) and (9) are obtained.

The silencer 40 of the CPAP device 10 does not have to include the second noise cancellation chamber 42B. As long as the first noise cancellation chamber 42A is arranged, advantage (1) is obtained.

The silencer 40 of the CPAP device 10 may have three or more noise cancellation chambers. Furthermore, the silencer 40 of the CPAP device 10 may include a noise cancellation chamber between the first flow passage 41A and the second flow passage 41B in a direction parallel to the second axis Y. In this case, the wall 44 that separates the noise cancellation chamber and the flow passage 41 may include any number of openings at any locations. Preferably, an opening is included in the wall 44 that separates the noise cancellation chamber and the first connection passage 41D.

The second opening 45B may be included in the wall 44 that separates the second noise cancellation chamber 42B and the second flow passage 41B, instead of the wall 44 that separates the second noise cancellation chamber 42B and the third flow passage 41C. Alternatively, the second opening 45B may be included in the wall 44 that separates the second noise cancellation chamber 42B and the third flow passage 41C, and another opening may be included in the wall 44 that separates the second noise cancellation chamber 42B and the second flow passage 41B. Preferably, an opening is arranged in at least one of the wall 44 that separates the second noise cancellation chamber 42B and the second flow passage 41B and the wall 44 that separates the second noise cancellation chamber 42B and the third flow passage 41C.

The third flow passage 41C may be located farther from the blower 20 than the second flow passage 41B is with respect to the airflow direction. Specifically, for example, the following structure may be adopted. One end of the third flow passage 41C is connected to the second end of the first inflow passage 51. The second flow passage 41B is located at a side of the third flow passage 41C in the second positive direction Y1, and the first flow passage 41A is located at a side of the second flow passage 41B in the second positive direction Y1. One end of the first flow passage 41A is connected to the first end of the first outflow passage 52. The second connection passage 41E connects the other end of the third flow passage 41C and one end of the second flow passage 41B. The first connection passage 41D connects the other end of the second connection passage 41E and the other end of the first flow passage 41A.

Even with such a structure, in which the second connection passage 41E is located farther from the blower 20 than the first connection passage 41D is with respect to the airflow direction, advantage (1) is obtained.

Also, even with this structure, in which the third flow passage 41C is located farther from the blower 20 than the second flow passage 41B is with respect to the airflow direction, and the wall 44 that separates the second noise cancellation chamber 42B and the third flow passage 41C includes the second opening 45B, advantages (1) and (9) are obtained.

The silencer 40 of the CPAP device 10 does not have to include the sound absorbing material 43 arranged in the first noise cancellation chamber 42A. Even if the sound absorbing material 43 is not arranged, the noise cancellation effect is improved by the shape of the flow passage 41 and formation of the first noise cancellation chamber 42A. The same applies to the second noise cancellation chamber 42B.

The material of the sound absorbing material 43 is not limited to the example described in the above embodiment. For example, the material of the sound absorbing material 43 may include polyester, glass wool, felt, or the like. The sound absorbing material 43 may be any porous material.

The first opening 45A does not have to be closed by the film F. Even in this case, advantage (1) is obtained. Furthermore, the second opening 45B does not have to be closed by the film F. Even in this case, advantages (1) and (9) are obtained.

The material of the film F is not limited to the example described in the above embodiment. For example, the film F may be made of paper.

### Clauses

Technical concepts obtained from the above embodiment and the modifications will now be described.

(1) A silencer for a CPAP device, the silencer including: a flow passage through which air flows in accordance with operation of a blower; and a noise cancellation chamber that allows for transmission of pressure fluctuations of the air in the flow passage, in which: the flow passage includes a first flow passage extending along a first axis, a second flow passage extending along the first axis and arranged next to the first flow passage in a direction orthogonal to the first axis, a third flow passage extending along the first axis and arranged next to the second flow passage in the direction orthogonal to the first axis, a first connection passage connecting an end of the first flow passage in a positive direction and an end of the second flow passage in the positive direction, the positive direction being one of two directions parallel to the first axis, and a second connection passage connecting an end of the second flow passage in a negative direction and an end of the third flow passage in the negative direction, the negative direction being another one of the two directions parallel to the first axis and being opposite to the positive direction; a maximum dimension of the first connection passage in a direction in which the second flow passage is arranged next to the third flow passage differs from a maximum dimension of the second connection passage in the direction in which the second flow passage is arranged next to the third flow passage; the noise cancellation chamber is located between the second flow passage and the third flow passage in the direction in which the second flow passage is arranged next to the third flow passage; and the noise cancellation chamber is connected to the second connection passage through an opening included in a wall that separates the noise cancellation chamber and the second connection passage.

(2) The silencer according to clause (1), further including a sound absorbing material arranged in the noise cancellation chamber.

(3) The silencer according to clause (1) or (2), in which the noise cancellation chamber includes an inner surface on which an opposing surface is defined, the opposing surface opposing an opening surface of the opening and being non-perpendicular with respect to a direction orthogonal to the opening surface.

(4) The silencer according to any one of clauses (1) to (3), in which a maximum dimension of the noise cancellation chamber in a direction parallel to the first axis is greater than a maximum dimension of the second connection passage in the direction parallel to the first axis, and a maximum dimension of the noise cancellation chamber in the direction in which the second flow passage is arranged next to the third flow passage is greater than each of a maximum dimension of the second flow passage in the direction in which the second flow passage is arranged next to the third flow passage and a maximum dimension of the third flow passage in the direction in which the second flow passage is arranged next to the third flow passage.

(5) The silencer according to any one of clauses (1) to (4), in which the second connection passage is located closer to the blower than the first connection passage is with respect to an airflow direction.

(6) The silencer according to any one of clauses (1) to (5), in which an open area of the opening is greater than a cross-sectional flow area of the second connection passage.

(7) The silencer according to any one of clauses (1) to (6), in which a maximum dimension of the opening in the direction in which the second flow passage is arranged next to the third flow passage is less than a maximum dimension of the noise cancellation chamber in the direction in which the second flow passage is arranged next to the third flow passage.

(8) The silencer according to any one of clauses (1) to (7), further including: a film thinner than the wall that separates the noise cancellation chamber and the second connection passage, in which the film closes the opening.

(9) The silencer according to any one of clauses (1) to (8), further including: when the noise cancellation chamber is a first noise cancellation chamber, and the opening that connects the first noise cancellation chamber and the second connection passage is a first opening, a second noise cancellation chamber that allows for transmission of pressure fluctuations of the air in the flow passage, in which the second noise cancellation chamber is located between the second flow passage and the third flow passage in the direction in which the second flow passage is arranged next to the third flow passage, and the second noise cancellation chamber is connected to the flow passage through a second opening included in a wall that separates the second noise cancellation chamber and the flow passage.

(10) The silencer according to clause (9), in which the second opening is included in a wall that separates the second noise cancellation chamber and the second flow passage or a wall that separates the second noise cancellation chamber and the third flow passage.

(11) The silencer according to clause (10), in which the third flow passage is located closer to the blower than the second flow passage is with respect to an airflow direction, and the wall that separates the second noise cancellation chamber and the third flow passage includes the second opening.

(12) A CPAP device, including: a blower that blows air; and a silencer, in which: the silencer includes a flow passage through which air flows in accordance with operation of the blower, and a noise cancellation chamber that allows for transmission of pressure fluctuations of the air in the flow passage; the flow passage includes a first flow passage extending along a first axis, a second flow passage extending along the first axis and arranged next to the first flow passage in a direction orthogonal to the first axis, a third flow passage extending along the first axis and arranged next to the second flow passage in the direction orthogonal to the first axis, a first connection passage connecting an end of the first flow passage in a positive direction and an end of the second flow passage in the positive direction, the positive direction being one of two directions parallel to the first axis, and a second connection passage connecting an end of the second flow passage in a negative direction and an end of the third flow passage in the negative direction, the negative direction being another one of the two directions parallel to the first axis and being opposite to the positive direction; a maximum dimension of the first connection passage in a direction in which the second flow passage is arranged next to the third flow passage differs from a maximum dimension of the second connection passage in the direction in which the second flow passage is arranged next to the third flow passage; the noise cancellation chamber is located between the second flow passage and the third flow passage in the direction in which the second flow passage is arranged next to the third flow passage; and the noise cancellation chamber is connected to the second connection passage through an opening included in a wall that separates the noise cancellation chamber and the second connection passage.

(13) The CPAP device according to clause (12), in which the second connection passage is located closer to the blower than the first connection passage is with respect to an airflow direction.

(14) The CPAP device according to clause (12) or (13), further including: when the noise cancellation chamber is a first noise cancellation chamber, and the opening that connects the first noise cancellation chamber and the second connection passage is a first opening, a second noise cancellation chamber that allows for transmission of pressure fluctuations of the air in the flow passage, in which the second noise cancellation chamber is located between the second flow passage and the third flow passage in the direction in which the second flow passage is arranged next to the third flow passage, the second noise cancellation chamber is connected to the flow passage through a second opening included in a wall that separates the second noise cancellation chamber and the third flow passage, and the third flow passage is located closer to the blower than the second flow passage is with respect to the airflow direction.
10) CPAP device
MU) main unit
SU) sub-unit
40) silencer
44) wall
41) flow passage
41A) first flow passage
41B) second flow passage
41BW) second flow passage width
41C) third flow passage
41CW) third flow passage width
41D) first connection passage
41DL) first connection passage length
41E) second connection passage
41EL) second connection passage length
41EW) second connection passage width
42A) first noise cancellation chamber
SW) first noise cancellation chamber width
SL) first noise cancellation chamber length
42B) second noise cancellation chamber
45A) first opening
45AW) first opening width
45B) second opening
43) noise absorbing material
OS) opposing surface
F) film

1. A silencer for a CPAP device, the silencer comprising:
a flow passage through which air flows in accordance with operation of a blower; and
a noise cancellation chamber configured to allow for transmission of pressure fluctuations of the air in the flow passage, wherein:
the flow passage includes
a first flow passage extending along a first axis,
a second flow passage extending along the first axis and arranged next to the first flow passage in a direction orthogonal to the first axis,
a third flow passage extending along the first axis and arranged next to the second flow passage in the direction orthogonal to the first axis,
a first connection passage connecting an end of the first flow passage in a positive direction and an end of the second flow passage in the positive direction, the positive direction being one of two directions parallel to the first axis, and
a second connection passage connecting an end of the second flow passage in a negative direction and an end of the third flow passage in the negative direction, the negative direction being another one of the two directions parallel to the first axis and being opposite to the positive direction;
a maximum dimension of the first connection passage in a direction in which the second flow passage is arranged next to the third flow passage differs from a maximum dimension of the second connection passage in the direction in which the second flow passage is arranged next to the third flow passage;
the noise cancellation chamber is located between the second flow passage and the third flow passage in the direction in which the second flow passage is arranged next to the third flow passage; and
the noise cancellation chamber is connected to the second connection passage through an opening included in a wall separating the noise cancellation chamber and the second connection passage.

2. The silencer according to claim 1, further comprising a sound absorbing material arranged in the noise cancellation chamber.

3. The silencer according to claim 1, wherein the noise cancellation chamber includes an inner surface on which an opposing surface is defined, the opposing surface opposing an opening surface of the opening and being non-perpendicular with respect to a direction orthogonal to the opening surface.

4. The silencer according to claim 1, wherein
a maximum dimension of the noise cancellation chamber in a direction parallel to the first axis is greater than a maximum dimension of the second connection passage in the direction parallel to the first axis, and
a maximum dimension of the noise cancellation chamber in the direction in which the second flow passage is arranged next to the third flow passage is greater than each of a maximum dimension of the second flow passage in the direction in which the second flow passage is arranged next to the third flow passage and a maximum dimension of the third flow passage in the direction in which the second flow passage is arranged next to the third flow passage.

5. The silencer according to claim 1, wherein the second connection passage is located closer to the blower than the first connection passage is with respect to an airflow direction.

6. The silencer according to claim 1, wherein an open area of the opening is greater than a cross-sectional flow area of the second connection passage.

7. The silencer according to claim 1, wherein a maximum dimension of the opening in the direction in which the second flow passage is arranged next to the third flow passage is less than a maximum dimension of the noise cancellation chamber in the direction in which the second flow passage is arranged next to the third flow passage.

8. The silencer according to claim 1, further comprising:
a film thinner than the wall separating the noise cancellation chamber and the second connection passage,
wherein the film closes the opening.

9. The silencer according to claim 1, further comprising:
when the noise cancellation chamber is a first noise cancellation chamber, and the opening connecting the first noise cancellation chamber and the second connection passage is a first opening, a second noise cancellation chamber configured to allow for transmission of pressure fluctuations of the air in the flow passage, wherein
the second noise cancellation chamber is located between the second flow passage and the third flow passage in the direction in which the second flow passage is arranged next to the third flow passage, and
the second noise cancellation chamber is connected to the flow passage through a second opening included in a wall separating the second noise cancellation chamber and the flow passage.

10. The silencer according to claim 9, wherein the second opening is included in a wall separating the second noise cancellation chamber and the second flow passage or a wall separating the second noise cancellation chamber and the third flow passage.

11. The silencer according to claim 10, wherein
the third flow passage is located closer to the blower than the second flow passage is with respect to an airflow direction, and
the wall separating the second noise cancellation chamber and the third flow passage includes the second opening.

12. A CPAP device, comprising:
a blower configured to blow air; and
a silencer, wherein:
the silencer includes
a flow passage through which air flows in accordance with operation of the blower, and
a noise cancellation chamber configured to allow for transmission of pressure fluctuations of the air in the flow passage;
the flow passage includes
a first flow passage extending along a first axis,
a second flow passage extending along the first axis and arranged next to the first flow passage in a direction orthogonal to the first axis,
a third flow passage extending along the first axis and arranged next to the second flow passage in the direction orthogonal to the first axis,
a first connection passage connecting an end of the first flow passage in a positive direction and an end of the second flow passage in the positive direction, the positive direction being one of two directions parallel to the first axis, and
a second connection passage connecting an end of the second flow passage in a negative direction and an end of the third flow passage in the negative direction, the negative direction being another one of the two directions parallel to the first axis and being opposite to the positive direction;
a maximum dimension of the first connection passage in a direction in which the second flow passage is arranged next to the third flow passage differs from a maximum dimension of the second connection passage in the direction in which the second flow passage is arranged next to the third flow passage;
the noise cancellation chamber is located between the second flow passage and the third flow passage in the direction in which the second flow passage is arranged next to the third flow passage; and
the noise cancellation chamber is connected to the second connection passage through an opening included in a wall separating the noise cancellation chamber and the second connection passage.

13. The CPAP device according to claim 12, wherein the second connection passage is located closer to the blower than the first connection passage is with respect to an airflow direction.

14. The CPAP device according to claim 13, further comprising:
when the noise cancellation chamber is a first noise cancellation chamber, and the opening connecting the first noise cancellation chamber and the second connection passage is a first opening, a second noise cancellation chamber configured to allow for transmission of pressure fluctuations of the air in the flow passage, wherein
the second noise cancellation chamber is located between the second flow passage and the third flow passage in the direction in which the second flow passage is arranged next to the third flow passage,
the second noise cancellation chamber is connected to the flow passage through a second opening included in a wall separating the second noise cancellation chamber and the third flow passage, and
the third flow passage is located closer to the blower than the second flow passage is with respect to the airflow direction.

15. The silencer according to claim 2, wherein the noise cancellation chamber includes an inner surface on which an opposing surface is defined, the opposing surface opposing an opening surface of the opening and being non-perpendicular with respect to a direction orthogonal to the opening surface.

16. The silencer according to claim 2, wherein
a maximum dimension of the noise cancellation chamber in a direction parallel to the first axis is greater than a maximum dimension of the second connection passage in the direction parallel to the first axis, and
a maximum dimension of the noise cancellation chamber in the direction in which the second flow passage is arranged next to the third flow passage is greater than each of a maximum dimension of the second flow passage in the direction in which the second flow passage is arranged next to the third flow passage and a maximum dimension of the third flow passage in the direction in which the second flow passage is arranged next to the third flow passage.

17. The silencer according to claim 3, wherein
a maximum dimension of the noise cancellation chamber in a direction parallel to the first axis is greater than a maximum dimension of the second connection passage in the direction parallel to the first axis, and
a maximum dimension of the noise cancellation chamber in the direction in which the second flow passage is arranged next to the third flow passage is greater than each of a maximum dimension of the second flow passage in the direction in which the second flow passage is arranged next to the third flow passage and a maximum dimension of the third flow passage in the direction in which the second flow passage is arranged next to the third flow passage.

18. The silencer according to claim 2, wherein the second connection passage is located closer to the blower than the first connection passage is with respect to an airflow direction.

19. The silencer according to claim 3, wherein the second connection passage is located closer to the blower than the first connection passage is with respect to an airflow direction.

20. The silencer according to claim 4, wherein the second connection passage is located closer to the blower than the first connection passage is with respect to an airflow direction.
